(19) United States
(12) Patent Application Publication
(10) Pub. No.: US 2025/0325789 A1
(43) Pub. Date: Oct. 23, 2025

(54) CONTROL AND METHOD FOR PARTIAL OCCLUSION OF A VESSEL

(71) Applicant: Prytime Medical Devices, Inc., Boerne, TX (US)

(72) Inventors: Keith P. MASLIN, Boerne, TX (US); Gregory S. SCHMD, Boerne, TX (US); Mathew Charles PICKERING, Boerne, TX (US); Todd J. KRUMMENACHER, Boerne, TX (US); Lincoln M. HARRIS, Boerne, TX (US); Rico JAEGER, Boerne, TX (US); Curtis J. FRANKLIN, Boerne, TX (US)

(21) Appl. No.: 18/866,850

(22) PCT Filed: May 17, 2023
(86) PCT No.: PCT/US2023/022573

§ 371 Date: Nov. 18, 2024

Related U.S. Application Data
(60) Provisional application No. 63/342,836, filed on May 17, 2022.

(51) Int. Cl. A61M 25/10; G16H 40/63

(52) U.S. Cl. CPC A61M 25/10187; A61M 25/10181; G16H 40/63; A61M 2205/10; A61M 2205/15; A61M 2205/18; A61M 2205/3317; A61M 2205/3327; A61M 2205/3331; A61M 2205/50; A61M 2210/12; A61M 2230/005; A61M 2230/30

(57) ABSTRACT

A control system for an occlusion catheter having a balloon includes a fluid reservoir containing a fluid and a fluid connector connectable to the reservoir and to the catheter to provide fluid communication therebetween. The fluid connector has a pressure sensor integrated therein. A pump is configured to move the fluid between the reservoir and the balloon via the fluid connector. A controller is in electrical communication with the pump and the pressure sensor. The controller is configured to: (i) receive data from the pressure sensor, the data being indicative of a pressure within the balloon, and (ii) in an autonomous mode of operation, control the pump to alter a size of the balloon by driving fluid from the reservoir toward the balloon or withdrawing fluid from the balloon, the control of the pump being based, at least in part, on the data from the pressure sensor.

### BACKGROUND OF THE INVENTION

The disclosure relates to controls and related methods for occlusion catheters and, more particularly, to vascular occlusion catheters capable of performing both partial and full vascular occlusion. The preferred controls and methods relate to controlling partial occlusion of a patient's aorta in a resuscitative endovascular balloon occlusion of the aorta (“REBOA”) procedure.

Vascular occlusion may be indicated in either the venous system and/or the arterial system. Endoarterial occlusion, such as REBOA, is a procedure in which a blood vessel is at least partially occluded in order to restrict blood flow upstream or downstream of the occlusion site for purposes of a vascular procedure or repair. Partial resuscitative endovascular balloon occlusion of the aorta (“P-REBOA”) is beneficial to mitigate the risk of ischemia below the site of the occlusion to limit or eliminate lack of blood flow to organs and tissue below the occlusion location. That is, partial perfusion past the occlusion balloon can provide the benefits of focusing or directing a majority of blood flow to the brain, heart and lungs or other upstream organs and tissue of the patient, but also potentially increasing the amount of time the occlusion balloon can be implanted in the patient, by providing at least partial blood flow to the patient's organs downstream of the occlusion member, such as to the patient's liver, digestive tract, kidneys and legs. Prior art systems have encountered difficulty during P-REBOA procedures in controlling the flow of blood past the occlusion balloon based on the dynamic nature of the patient's circulatory system, difficulty measuring the amount of blood flow that bypasses the occlusion balloon and many additional factors.

It would, therefore, be desirable to further design, develop and implement an occlusion balloon catheter configured to partially occlude the target blood vessel while permitting partial perfusion to the patient's organs downstream thereof. It would be particularly desirable to design, develop and implement a catheter system that is controllable to maintain partial occlusion of a patient's aorta in a certain target range while adjusting along with the dynamics of patient resuscitation, to provide warnings or alarms to medical personnel when patient medical events are detected, and to guide the medical personnel's decision making and actions during the P-REBOA procedure.

### BRIEF SUMMARY OF THE INVENTION

Briefly stated, one embodiment comprises a control system for an occlusion catheter having an occlusion balloon to control partial occlusion of a vessel of a patient. The control system includes a fluid reservoir containing a fluid for use with the occlusion catheter and a fluid connector connectable to the fluid reservoir and to the occlusion catheter to provide fluid communication between the fluid reservoir and the occlusion catheter. The fluid connector has a pressure sensor integrated therein. A pump is configured to move the fluid between the fluid reservoir and the occlusion balloon via the fluid connector. A controller is in electrical communication with the pump and the pressure sensor of the fluid connector. The controller is configured to: (i) receive data from the pressure sensor of the fluid connector, the data being indicative of a pressure within the occlusion balloon, and (ii) in an autonomous mode of operation, control the pump to alter a size of the occlusion balloon by driving fluid from the fluid reservoir toward the occlusion balloon or withdrawing fluid from the occlusion balloon toward the fluid reservoir, the control of the pump being based, at least in part, on the data from the pressure sensor of the fluid connector.

In one aspect, the controller is further configured to receive data representing a physiological value from at least one sensor located on or in the patient, and in the autonomous mode of operation, the control of the pump is further based on the received data and includes comparing the received data with a setpoint value or target range such that the size of the occlusion balloon is altered to cause the physiological value to be within a tolerance limit of the setpoint value or within the target range. In a further aspect, the physiological value is one of blood pressure upstream of the occlusion balloon or blood pressure downstream of the occlusion balloon. In a still further aspect, the blood pressure upstream of the occlusion balloon is one of pulse pressure, systolic blood pressure, or mean arterial pressure and the blood pressure downstream of the occlusion balloon is one of pulse pressure, systolic blood pressure, or mean arterial pressure.

In another aspect, the control of the pump includes detecting a leak in the occlusion balloon. The controller is configured to output an alert when the controller determines the pressure in the balloon has unintentionally decreased at least once during operation based on the data from the pressure sensor of the fluid connector. In a further aspect, the controller is configured to output the alert when the controller determines the pressure in the balloon has unintentionally decreased more than twice during operation.

In yet another aspect, the control of the pump includes stopping the pump if the data from the pressure sensor of the fluid connector indicates the pressure in the balloon exceeds a predetermined safety value.

In still another aspect, the control of the pump includes withdrawing fluid from the occlusion balloon in response to a command to empty the occlusion balloon. The controller is configured to stop the pump when the data from the pressure sensor of the fluid connector indicates a vacuum has been pulled in the occlusion balloon.

Another embodiment comprises a control system for an occlusion catheter having an occlusion balloon to control partial occlusion of a vessel of a patient. The control system includes a fluid reservoir containing a fluid for use with the occlusion catheter, a fluid connector connectable to the fluid reservoir and to the occlusion catheter to provide fluid communication between the fluid reservoir and the occlusion catheter, and a pump configured to move the fluid between the fluid reservoir and the occlusion balloon via the fluid connector. The pump has a pump housing including a pump lid and a pump body. The pump lid is movable with respect to the pump body between an open position and a closed position. A portion of the fluid connector is received within the pump housing. A controller is in electrical communication with the pump and the pressure sensor of the fluid connector. The controller is configured to: (i) enter an autonomous mode of operation when the pump lid and pump body are in the closed position, (ii) in the autonomous mode of operation, receive data representing a physiological value from at least one sensor located on or in the patient, compare the received data with a setpoint value or target range, and control the pump to alter the size of the occlusion balloon to cause the physiological value to be within a tolerance limit of the setpoint value or within the target range, (iii) enter a manual mode of operation when the pump lid and pump body are in the open position, and (iv) in the manual mode of operation, prevent operation of the pump to enable movement of the fluid between the fluid reservoir and the occlusion balloon while the fluid connector is installed in the pump housing.

In one aspect, the physiological value is one of blood pressure upstream of the occlusion balloon or blood pressure downstream of the occlusion balloon. In a further aspect, the blood pressure upstream of the occlusion balloon is one of pulse pressure, systolic blood pressure, or mean arterial pressure and the blood pressure downstream of the occlusion balloon is one of pulse pressure, systolic blood pressure, or mean arterial pressure.

In another aspect, the control system further includes a control hub housing. The controller is disposed within the control hub housing. In a further aspect, the pump is attached to the control hub housing.

In yet another aspect, the fluid reservoir is a syringe.

In still another aspect, the pump body includes a Hall sensor and the pump lid includes a magnet. The controller is configured to determine the pump lid and pump body are in the closed position upon receiving a signal from the Hall sensor that indicates the presence of the magnet.

In another aspect, the controller is further configured to, upon determining from the data from the at least one sensor indicates no physiological response to a change in size of the occlusion balloon, output an alert.

### BRIEF DESCRIPTION OF THE SEVERAL VIEWS OF THE DRAWINGS

The foregoing summary, as well as the following detailed description of preferred embodiments of the instrument, implant and method of the present application, will be better understood when read in conjunction with the appended drawings. For the purposes of illustrating the occlusion catheter, control hub and related controls and methods for P-REBOA, there are shown in the drawings preferred embodiments. It should be understood, however, that the application is not limited to the precise arrangements and instrumentalities shown. In the drawings:

FIG. 1 is a schematic block diagram of a control hub in use with an occlusion catheter in accordance with a first embodiment;

FIG. 2 is a front, left side perspective view of the control hub of FIG. 1;

FIG. 3 is a front side elevational view of the control hub of FIG. 1;

FIG. 4 is a front, bottom side perspective view of the control hub of FIG. 1;

FIG. 5 is a front, right side perspective view of the control hub of FIG. 1;

FIG. 6 is a rear, right side perspective view of the control hub of FIG. 1;

FIG. 7 is a top plan view of medical tubing for use with the control hub of FIG. 1;

FIG. 8 is a front, left side perspective view of the control hub of FIG. 1 with the pump lid open and the tubing installed;

FIG. 9 is a schematic block diagram of the control hub of FIG. 1 in use with a catheter and external vital sign monitor;

FIG. 10 is a screenshot from the display of the control hub of FIG. 1;

FIG. 11 is a schematic block diagram of an electro-mechanical system that may be utilized with the control hub of FIG. 1;

FIG. 12 is a flow chart of a process for balloon deflation that may be carried out by the control hub of FIG. 1;

FIG. 13 is a flow chart of a blood pressure control loop process that may be carried out by the control hub of FIG. 1;

FIG. 14 is a flow chart of a process for full aorta occlusion that may be carried out by the control hub of FIG. 1;

FIG. 15 is a flow chart of a process for balloon leak detection that may be carried out by the control hub of FIG. 1;

FIG. 16 is a flow chart of a process for determining a condition of no physiological response during balloon inflation that may be carried out by the control hub of FIG. 1;

FIG. 17 is a flow chart of a process for motor stall detection that may be carried out by the control hub of FIG. 1;

FIG. 18 is a flow chart of a process for activating autonomous or manual control in response to detecting a state of the pump lid by the control hub of FIG. 1;

FIGS. 19A-19D are screenshots from a vital sign monitor showing progressive changes to pulse pressure during a catheter balloon deflation event; and

FIG. 20 is a plot demonstrating blood pressure changes for two different patients as a function of flow percentage.

### DETAILED DESCRIPTION OF THE INVENTION

Certain terminology is used in the following description for convenience only and is not limiting. Unless specifically set forth herein, the terms “a”, “an” and “the” are not limited to one element but instead should be read as meaning “at least one”. The words “right”, “left”, “lower” and “upper” designate directions in the drawings to which reference is made. The words “inwardly” or “distally” and “outwardly” or “proximally” refer to directions toward and away from, respectively, the patient's body, or the geometric center of the preferred occlusion catheter, control hub and related controls and methods for P-REBOA and related parts thereof. The words, “anterior”, “posterior”, “superior,” “inferior”, “lateral” and related words and/or phrases designate preferred positions, directions and/or orientations in the human body to which reference is made and are not meant to be limiting. The terminology includes the above-listed words, derivatives thereof and words of similar import.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

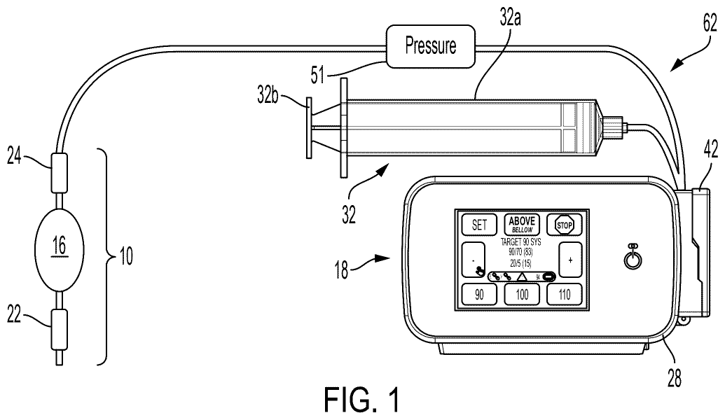

Referring to FIG. 1, a control hub 18 is shown connected to an occlusion catheter 10 in accordance with a first embodiment of the present disclosure. The catheter 10 may be designed and configured for the P-REBOA procedure and for controlling the blood flow during the P-REBOA procedure. The catheter 10 may include a first shaft/hypotube (not shown) that forms the structural backbone/chassis of the catheter 10, an atraumatic tip or P-tip (not shown) at a distal end of the catheter 10, an occlusion balloon 16, an above balloon or distal pressure sensor 22, and a below balloon or proximal pressure sensor 24.

The general structure and certain functions of the preferred catheter 10 are described in International Patent Application Publication No. WO 2022/197895, the entire contents of which are incorporated herein by reference. The herein described control system and method for partial occlusion of a vessel may also be utilized with any of the catheters and related features and methods described in International Patent Application Publication No. WO2022/016109, International Patent Application Publication No. WO 2020/033372, and U.S. Pat. No. 10,569,062, the entire contents of which are incorporated herein by reference in their entireties. The control system described herein is not limited to being utilized with the catheter 10 shown in the present disclosure or in the cited publications and may be utilized with nearly any catheter having an occlusion balloon for occluding or partially occluding a patient's vessel.

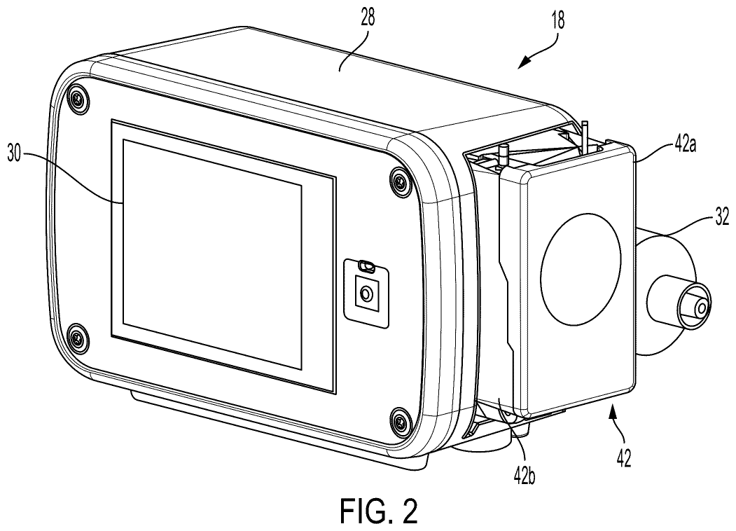

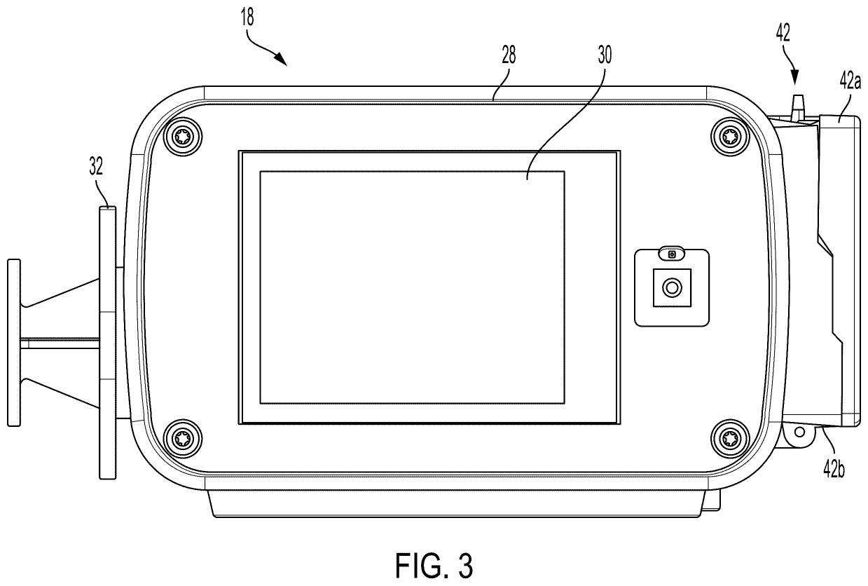

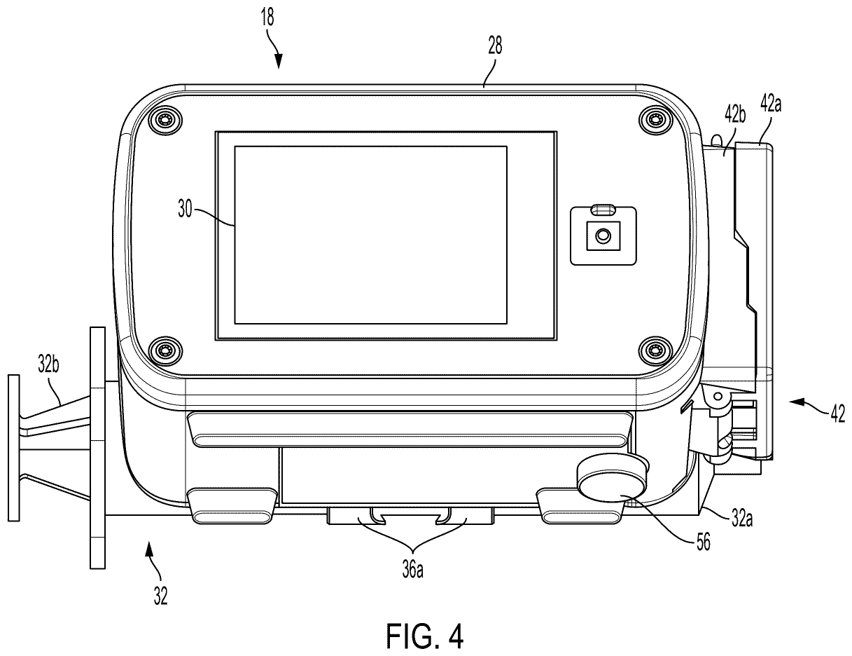

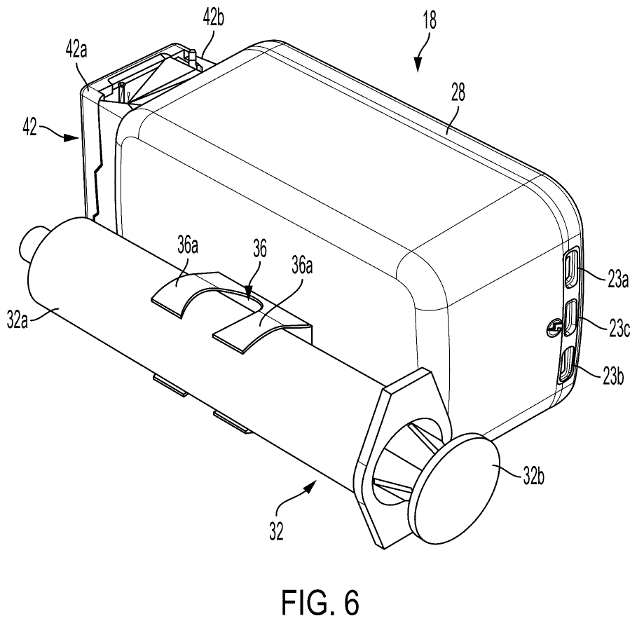

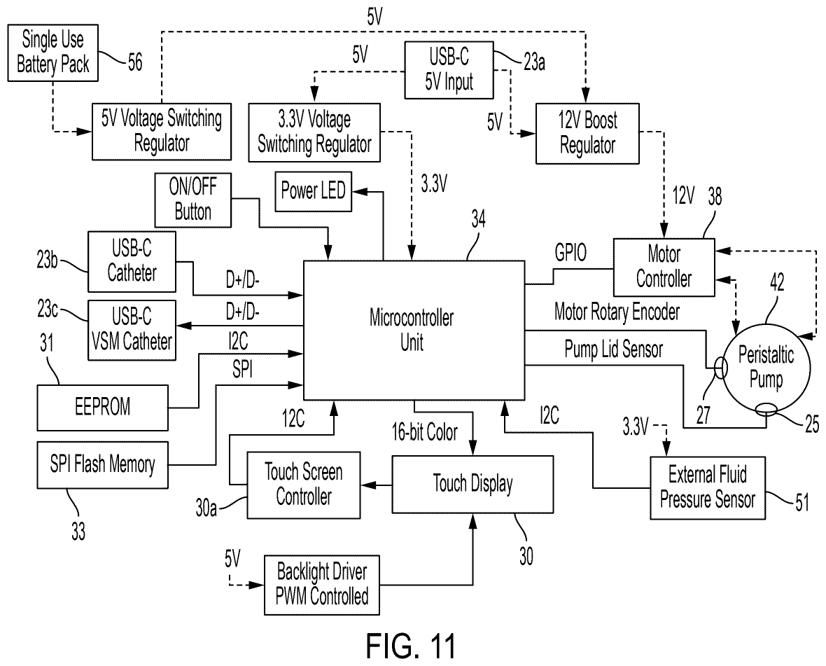

Referring to FIGS. 1-6, the control hub 18 may take on various forms and include various features, but preferably includes at least a display 30 that displays acquired data from the proximal and distal pressure sensors 24, 22. The display 30 may be a touchscreen display operable by a user to interactively select functions, as will be described in more detail below. In such embodiments, the control hub 18 may include a touchscreen controller 30a (FIG. 11) for detecting positional contact on the display 30. The control hub 18 may be configured to acquire data from the distal and proximal pressure sensors 22, 24, display the acquired data and control full and/or partial occlusion of the patient's vessel using the occlusion balloon 16 based on the acquired data.

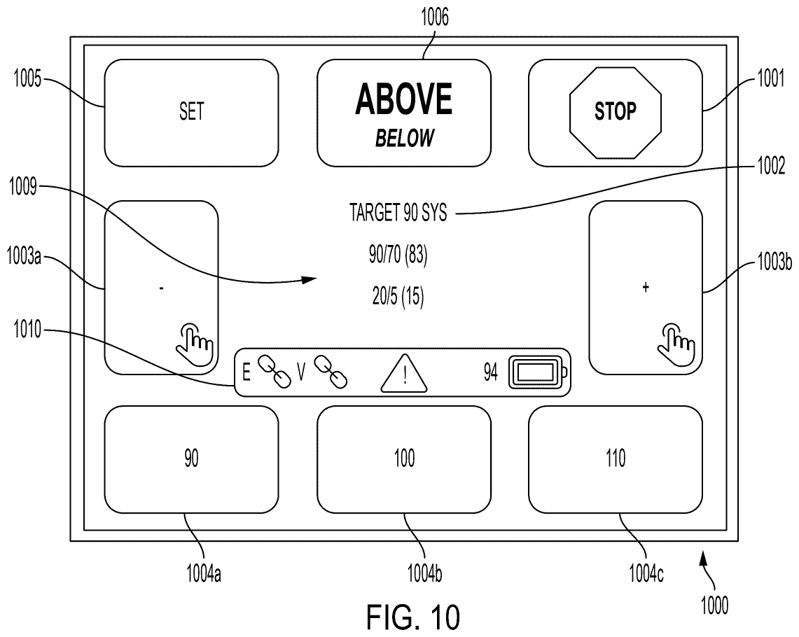

The control hub 18 may be in fluid communication with the occlusion balloon 16 and may take on several forms, but preferably includes a hub housing 28 and a hub display 30 disposed on or within the hub housing 28 to display procedure information to the user. The control hub 18 may be in further fluid communication with a fluid reservoir 32 that stores occlusion fluid for supply to the occlusion balloon 16 to facilitate inflation and deflation of the occlusion balloon 16 based on control signals from a controller 34 (FIG. 10) in the control hub 18. The occlusion fluid may be a prefilled sterile saline, saline/radiographic contrast solution, or gas media, but is not so limited and may be comprised of nearly any biocompatible fluid or material that is able to perform the functions of the fluid, withstand the normal operating conditions of the fluid and flow within the catheter 10 during normal operation. The biocompatible fluid may, for example, be comprised of a biocompatible gas such as carbon dioxide (CO₂), Helium or another biocompatible fluid/gas that facilitates inflation and/or deflation of the occlusion balloon 16.

The hub housing 28 may be constructed of a sturdy enclosure structure, which may or may not be sterilizable, that is able to take on the size and shape of the hub housing 28, withstand the normal operating conditions of the hub housing 28, and encase all the components required to perform the functions of the control hub 18. The hub housing 28 may be relatively small, e.g., handheld, and may be designed with ergonomic form factors to improve efficiency and comfort in the working environment for the patient and user. The hub housing 28 may be constructed of a strong, stiff, medical-grade structural material that is relatively non-absorbent that is able to take on the size and shape of the hub housing 28, withstand the normal operating conditions of the hub housing 28, and perform the intended functions of the hub housing 28. The hub housing 28 may be constructed of a polyethylene (“PE”) material, but is not so limited and may be otherwise designed and constructed to house the described components of the control hub 18. The hub housing 28 may alternatively be constructed of a lightweight metal housing, constructed of materials such as aluminum or magnesium-based materials. The hub housing 28 may include shock-absorbing and anti-slip materials positioned on edges, corners and adjacent surfaces to facilitate gripping of the hub housing 28 by the user or patient and to limit slipping of the hub housing 28 relative to the patient during use or when the catheter 10 is stored in a medical facility or by medical personnel. The shock-absorbing and anti-slip materials may be rubberized polymer or silicone to provide a shock-absorbent barrier and anti-slip feature. The control hub 18 may be re-usable and non-sterile, although sterile and single use versions are possible as well.

The control hub 18 may further accommodate removable mounting of the fluid reservoir 32 to the hub housing 28 at a reservoir slot 36, which is shown in the drawings as a pair of spring clip arms 36a that may releasably grasp the fluid reservoir 32 for attachment to the control hub 18. As shown in FIGS. 1-6, the fluid reservoir 32 may be in the form of a syringe having a syringe body 32a and a plunger 32b that is configured to move within the syringe body 32a. In addition to the automatic titration of the fluid from the syringe 32 that will be described in more detail below, a user may be able to manually manipulate the plunger 32b to drive fluid from the syringe body 32a into the balloon 16 or withdraw fluid from the balloon 16 into the syringe body 32a. The fluid reservoir 32 can alternatively be a bag, cartridge, or the like. The hub housing 28 may alternatively enclose a permanent reservoir, accommodate a removable reservoir, or combinations thereof, including use with a syringe 32 or other type of reservoir described above. The hub housing 28 is not limited to including the reservoir slot 36 and may include a connection, such as a Luer connection (not shown), that permits fluid connection of the hub housing 28 to the fluid reservoir 32 and/or the catheter 10, respectively. The hub housing 28 is shown in FIG. 1 as being a separate component from the catheter 10 that may be connected thereto via medical tubing 62 placing the syringe 32 in fluid communication with the balloon 16. However, in other embodiments, the hub housing 28 may be connected to the catheter 10 in other configurations or may be integrally assembled with the catheter 10.

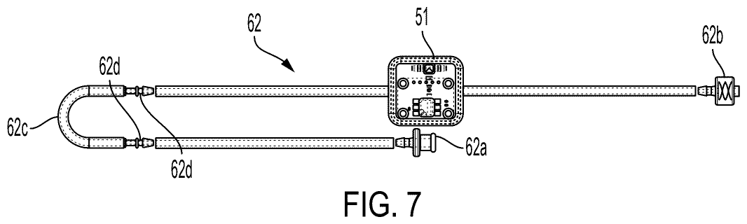

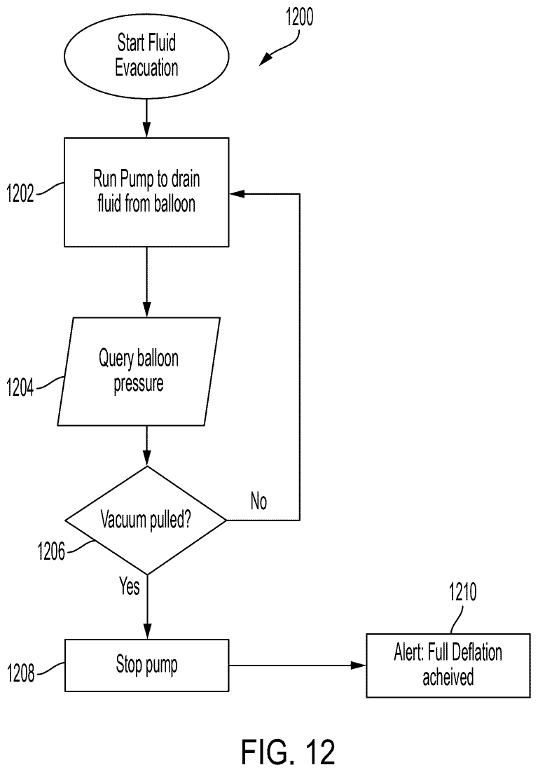

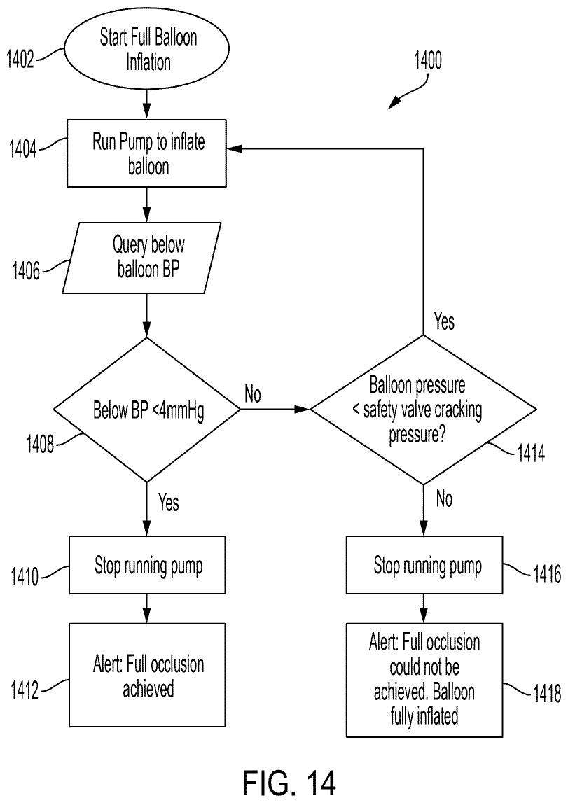

As explained above, the syringe 32 is shown being in fluid communication with the catheter 10 by the medical tubing 62. An example of the medical tubing 62 is shown in FIG. 7. The medical tubing 62 may include Luer locks 62a, 62b at opposing ends thereof for respective connection to the syringe 32 and to the catheter 10, although other types of connections may be used as well. Embodiments like that shown in FIGS. 1-7 allow a different syringe (not shown) to be used for initial insertion of the catheter 10 into the patient. When initial set-up is complete, the other syringe may be disconnected from the catheter 10 and the syringe 32 may be attached for maintaining desired blood pressures during subsequent procedures. As shown in FIGS. 1 and 7, the medical tubing 62 may include an integrated pressure sensor 51, preferably positioned downstream of the control hub 18, that acquires data indicative of pressure inside the occlusion balloon 16. The pressure sensor 51 may be a differential pressure sensor having two ports, such as the pressure sensor ABPMRRT030PD4A3 available from HONEYWELL. One port may measure atmospheric pressure while the other measures the pressure in the fluid line (medical tubing 62). The pressure sensor 51 may output the difference to a controller 34 (FIG. 11) of the control hub 18 or the like. An advantage of a differential pressure sensor is the self-correction with changing ambient conditions (such as in an emergency helicopter, or the like). Readings from the pressure sensor 51 may be used to prevent over-inflation of the balloon 16, which can waste inflation media. An example is described in further detail below with respect to FIG. 14, where inflation is stopped before pressure measured by the pressure sensor 51 reaches a safety valve cracking pressure. The pressure sensor 51 may also be used for determining when a vacuum has been pulled in the balloon 16 during deflation, as explained in further detail below with respect to FIG. 12.

Although the pressure sensor 51 has been described above as being a differential pressure sensor, other types of pressure sensors may be used as well. The pressure sensor 51 may be disposable so that the medical tubing 62 or portions thereof in contact with a patient may be discarded following usage with a patient, although it may be possible in some other embodiments to utilize a reusable pressure sensor 51 that may be sterilized between uses.

The medical tubing 62 may further include a pump tubing section 62c configured for installation in a pump 42 of the control hub 18, as will be described in further detail below. At least the pump tubing section 62c of the medical tubing 62 may be made from a compressible material, such as a thermoplastic elastomer, rubber, or other like materials. The pump tubing section 62c may further include connectors 62d, such as plastic nuts or the like, for attaching the pump tubing section 62c to a remainder of the medical tubing 62, which may be made from a more standard plastic or like material. However, larger portions of the medical tubing 62 may be made from the compressible material to allow for variation in placement of the medical tubing 62 with respect to the pump 42, or the entire medical tubing 62 may be made from the compressible material. In embodiments with a different pump style, the medical tubing 62 may also be entirely made from a standard plastic or like material. The materials for the medical tubing 62 may therefore be selected based on the type of pump 42 deployed and other expected operational conditions.

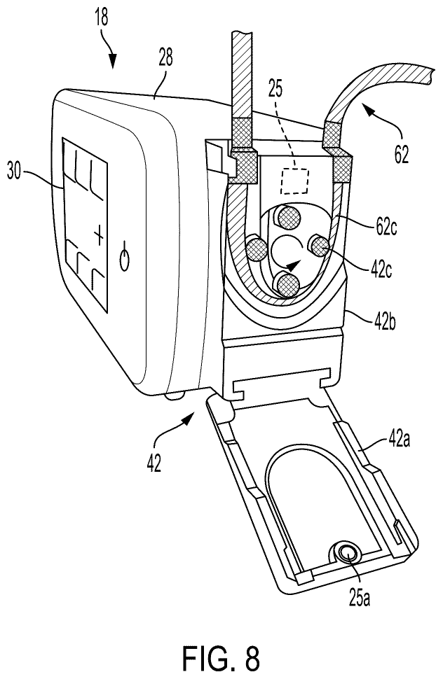

Referring to FIG. 8, the pump 42 may be a peristaltic pump, such as the 520 RPM 29QQ series pump available from BOXER, however the pump 42 is not limited to this type and may be any type of pump that can be used to move fluid, such as diaphragm, plunger or piston, gear, vane, nutating, volute, impeller, piezo, or other types. The pump 42 may include a pump lid 42a and a pump body 42b. The pump body 42b may house a plurality of rollers 42c configured to compress the pump tubing section 62c during rotation when in operation. The pump lid 42a may also be used in conjunction with the rollers 42c to mechanically secure and compress the pump tubing section 62c. For example, the pump lid 42a may be sized and shaped to, when the pump lid 42a is closed (e.g., FIGS. 1-4), sufficiently compress the pump tubing section 62c into the operational section of the pump 42 so that the rollers 42c can effect fluid movement in the medical tubing 62. However, when the pump lid 42a is open, as shown in FIG. 8, manual operation of the syringe 32 may be performed since the rollers 42c will express insufficient compression on the pump tubing section 62c. The pump 42 is shown in the figures as being attached to the hub housing 28, but the pump 42 may also be contained at least partially within the hub housing 28, or may be separate from the hub housing 28.

Referring to FIG. 11, the hub housing 28 may also enclose a controller 34, which in FIG. 11 is shown as a microcontroller unit (MCU), although the controller 34 may take other forms, such as a central processing unit (CPU), a microprocessor, an application specific controller (ASIC), a programmable logic array (PLA), combinations thereof, or the like. The controller 34 may include or be coupled to a memory (e.g., EEPROM 31, flash memory 33, or the like) that may store code or software for carrying out processes described herein and/or carrying out other operations of the control hub 18 and may store any captured data for later transfer to remote or external devices (e.g., smartphones, tablets, computers, or the like). It should be further appreciated that although controller 34 is referred to in this example as a single component, the controller 34 may include a plurality of individual devices, with control functions divided among the individual devices. The controller 34 may be wired or wirelessly connected to various components described herein as necessary for carrying out the operations and processes described herein.

The controller 34 may be in communication with a motor controller 38 that may be housed within the hub housing 28 and/or with the pump 42. The controller 34 may direct the motor controller 38 to operate a motor (not shown) of the pump 42 for driving or withdrawing fluid. The motor may be a stepper motor, a servo motor, or the like. The controller 34 may also be in communication with a motor encoder 27 to enable counting of motor revolutions. The controller 34 may store a known or predetermined value relating to the volume of fluid per revolution, and therefore be able to calculate, based on the readings from the motor encoder 27, the volume of fluid directed into or withdrawn from the balloon by the pump 42 based on the number of motor revolutions detected. The controller 34 may further be in communication with a pump lid sensor 25 (FIG. 8), which may be a Hall effect sensor, although other types of sensors may be used as well to detect whether the pump lid 42a is closed. For example, the pump lid sensor 25 may be mounted on or in the pump body 42b and a corresponding magnet 25a may be provided on the pump lid 42a. The pump lid sensor 25 may send a signal to the controller 34 regarding the status of the pump lid 42a based on, for example, whether the presence of the magnet 25a is detected or not.

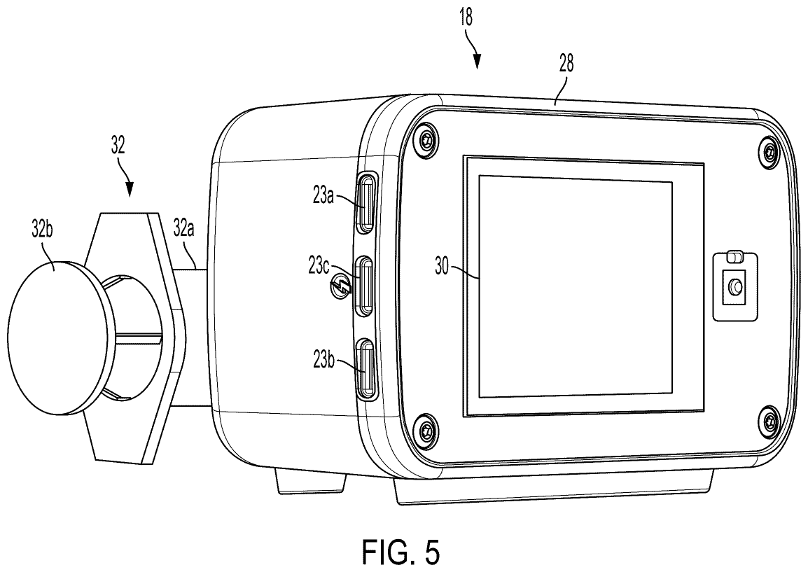

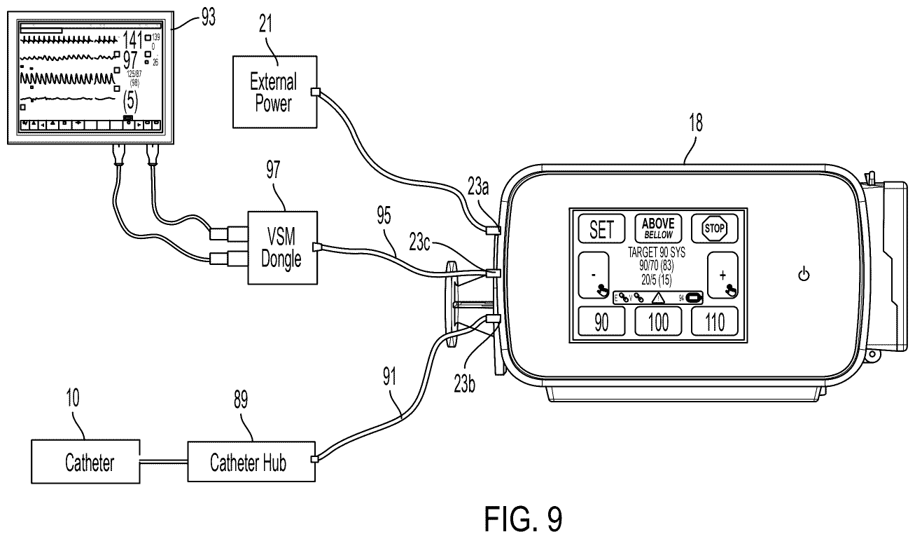

The control hub 18 may be powered by one or more batteries 56 that may be arranged in a battery pack removable and replaceable from the hub housing 28. The batteries 56 may be non-rechargeable, but the control hub 18 is not so limited and may include rechargeable batteries (not shown), a hard wired power connection, combinations thereof, or other power sources to provide power to the components of the control hub 18. For example, as seen in FIGS. 5 and 9, the control hub 18 may be provided with a USB-C port 23a or other similar type of port configured to allow connection to an external power source 21, such as an outlet, computer, or the like. In the absence of an external power source 21 or in embodiments where the control hub 18 does not enable external power connection, the batteries 56 may power the controller 34, motor controller 38, motor, pump 42, display 30 and related electrical components of the control hub 18.

The control hub 18 may be designed to function with the catheter 10 to support partial occlusion of a patient's vessel and, specifically, P-REBOA procedures. The fluid connection and metering of the occlusion fluid to and from the occlusion balloon 16 and the reservoir 32 can be accurately controlled by the control hub 18. The controller 34 may be designed and configured to collect data from the distal and proximal pressure sensors 22, 24, to control titration of balloon volume, and flow of blood past the occlusion balloon 16 in a P-REBOA procedure. The controller 34 may be able to allow the setting of target points to monitor and control partial occlusion, such as by setting a minimum blood pressure or blood pressure range monitored by the proximal pressure sensor 24 and/or a minimum blood pressure or blood pressure range monitored by the distal pressure sensor 22. Blood pressure values that can be measured, displayed, and used as setpoints for this purpose can be systolic blood pressure, diastolic blood pressure, mean arterial pressure (MAP), pulse pressure (difference between systolic and diastolic), combinations thereof, or the like.

Pulse pressure can especially provide a reliable method for predicting and/or regulating blood flow with closed loop control. At full occlusion in all patients, the distal blood pressure moves to the non-pulsatile range (systolic, diastolic and MAP are all approximately equal to each other) but not all patients achieve the same value. For example, at full occlusion, patient #1 might be 4, 4, 4, patient #2 might be 15, 15, 15, and patient #3 might be 35, 35, 35. If the user tried to control to a specific below-balloon setpoint for MAP of 14, the controller could deflate the balloon for patient #1 until a MAP of 14 was achieved. However, patients #2 and #3 would not be able to obtain a MAP of 14 below the balloon since their full occlusion blood pressure was higher than the setpoint. Due to this natural variation of full occlusion blood pressure below the balloon, a new variable may be preferred for closed loop control.

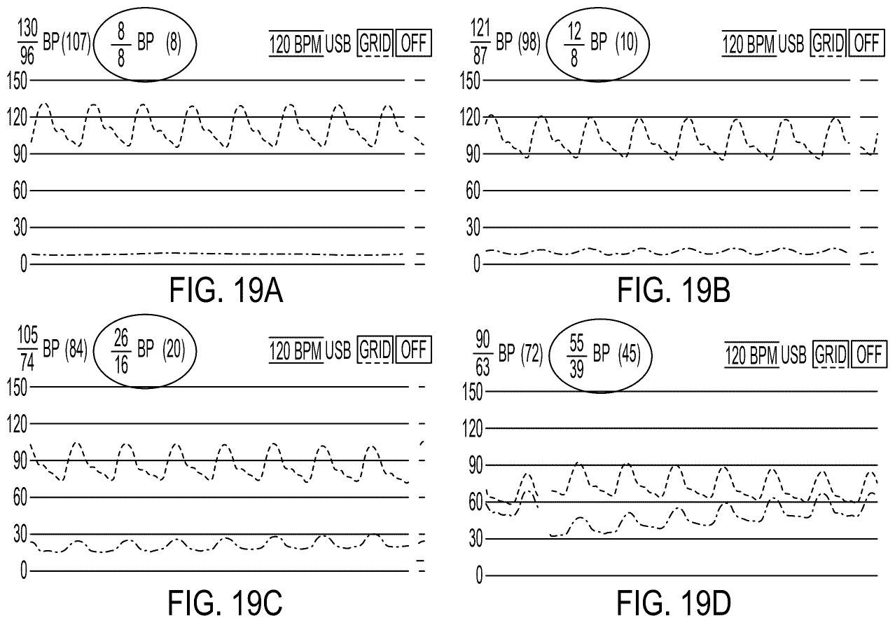

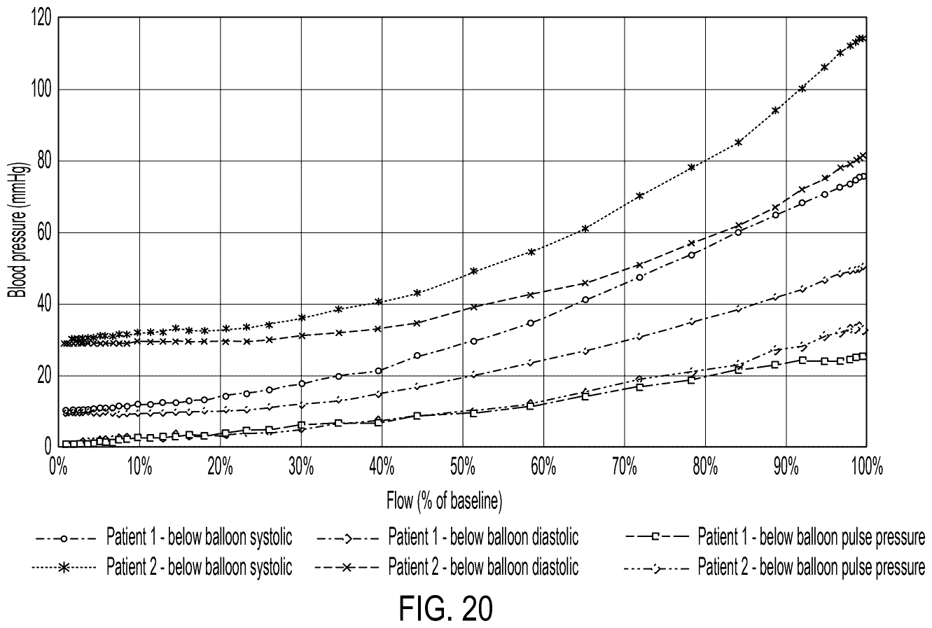

Applicant has observed in testing that patients achieve non-pulsatile blood pressure below the balloon at full occlusion. As the balloon is deflated, the pulsatility (i.e., pulse pressure) gradually starts to increase, as is shown in FIGS. 19A-19D and 20. If the controller allows the user to display and/or allow an input for pulse pressure, the user could target a desired pulse pressure setpoint or setpoint range that is achievable for all patients. For example, a pulse pressure of 0-5 mmHg would be achievable for all three of the previously identified patients. In FIG. 20, below-balloon systolic and diastolic pressures are plotted for two patients as a function of flow percentage, showing large and diverging differences as flow rate increases. However, the pulse pressure is also mapped for both patients, with the values for each remaining relatively consistent over the entire range of flow percentages.

FIG. 10 is an example screenshot 1000 from the control hub 18 shown in FIGS. 1-6. The display 30 may provide a selectable start/stop button 1001 for activating or deactivating autonomous operation. The appearance of the button 1001 may change depending on whether the controller 34 is operating in the autonomous mode or not. For example, in FIG. 10, a “STOP” sign is shown in the start/stop button 1001 because the controller 34 is currently operating in autonomous mode. The symbol may change when the controller 34 is allowing manual operation. In other embodiments, separate buttons for stopping and starting may be provided.

A selection button 1006 may be provided to allow a user to select whether to adjust a setpoint blood pressure above or below the balloon 16. In FIG. 10, the “ABOVE” option is selected, so the text for that option is emphasized. The current setpoint 1002 for the option selected (here the above-balloon blood pressure) may be displayed adjacent to current measurement data 1009, which in FIG. 10 provides both above-and below-balloon blood pressure readings. The user may utilize the adjustment keys 1003a, 1003b to respectively lower or raise the setpoint 1002 for the selected option. Alternatively, the display 30 may provide one or more preset options 1004a, 1004b, 1004c of predetermined setpoints that can be used to more quickly arrive at a desired setpoint. A “SET” button 1005 may also be provided for a user to select the current physiological state of the patient as a setpoint. In some embodiments, a long press of the positive adjustment key 1003b may cause the controller 34 to take the catheter 10 to a full occlusion state based on the below balloon blood pressure, which may result in the lowest amount of inflation necessary to obtain full occlusion. Similarly, a long press of the negative adjustment key 1003a may cause the controller 34 to perform a full evacuation of the balloon 16. The display 30 may further provide a status bar 1010 for notifications such as the connection status of connected devices (described further below), warning indications, battery life, or the like.

However, screenshot 1000 represents only one example embodiment of arrangement and functionality of the display 30 and other arrangements and implementations may be used as well. Confirmation screens for selections may be provided. Error messages may be displayed. Other items may be displayed as well, including timers, balloon 16 fill status, waveform graphs, and the like. Moreover, in some embodiments with or without a touchscreen display, mechanical buttons or hardware may be provided for making operational selections.

An example usage of the control hub 18 will now be described. The catheter 10 may be inserted into a patient's vessel, preferably the aorta, such that the occlusion balloon 16 is positioned at a desired level or zone in the vessel and the occlusion balloon 16 may then be inflated manually by the user. The initial manual inflation may be performed using the syringe 32 shown in FIGS. 1-6, or by a separate syringe or reservoir (not shown). In the latter instance, the initial syringe or reservoir may subsequently be detached from the catheter 10 (e.g., by removing a Luer connection or the like). The medical tubing 62 may then be coupled to the catheter 10 (e.g., by Luer lock 62b or the like). Any necessary electrical connections (such as to sensors 22, 24 or other components of the catheter 10) may be made as well, as needed. Manual control may continue with the control hub 18 attached, as desired, or autonomous control may be implemented. Under autonomous control, the controller 34 may direct the pump 42 to drive fluid from the reservoir 32 into the balloon 16 or withdraw fluid from the balloon 16 to maintain one or more physiological parameters (e.g., blood pressure, blood pH, lactate, potassium level, or the like) at a setpoint value. During autonomous control, the controller 34 may acquire data from the distal and proximal blood pressure sensors 22, 24, either directly or through an intermediate device such as a catheter hub 89 (FIG. 9 and as described in further detail below). The controller 34 may also acquire data from other sensors, as desired. The controller 34 may use the acquired data in comparison with the setpoint to adjust speed and/or rotation of the pump 42 as needed to achieve or maintain the physiological parameters at the desired setpoint.

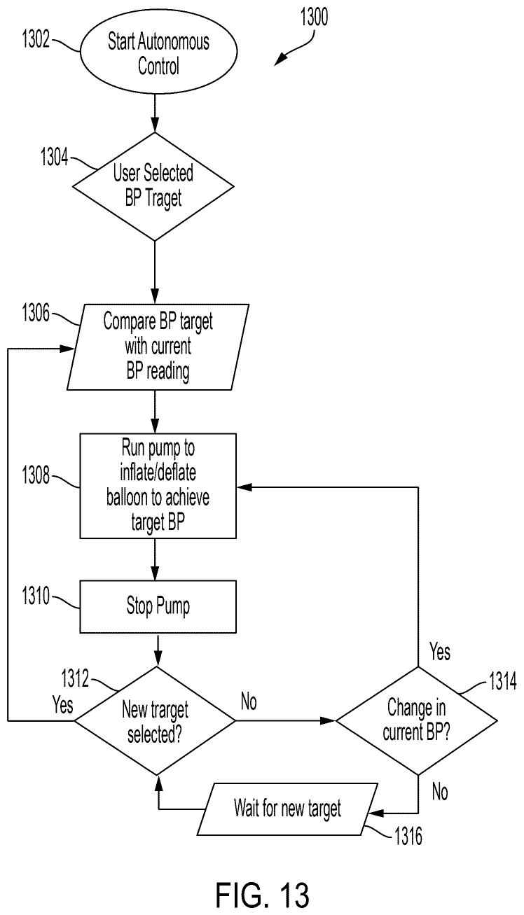

FIG. 13 shows an example method 1300 for execution by the controller 34 when autonomous control is selected for operation. At step 1302, the controller 34 may receive a command to autonomously maintain control of a blood pressure setpoint or setpoints. The command may be received from the display 30, such as from a selection by the user of the start/stop button 1001 or the SET button 1005 (FIG. 10), although other inputs to the control hub 18, including closure of the pump lid 42a, selection of other buttons or switches (not shown), or input from external devices, may be used as well. At step 1304, the controller 34 may receive a user-selected blood pressure setpoint, such as from the adjustment keys 1003a, 1003, presets 1004a-c, the SET button 1005 (FIG. 10), and/or other inputs to the control hub 18. At step 1306, the controller 34 may compare the setpoint to a current blood pressure reading provided by at least one of the blood pressure sensors 22, 24. At step 1308, the controller 34 may run the pump 42 to inflate or deflate the balloon 16 to achieve the blood pressure setpoint. When the setpoint is achieved, at step 1310, the controller 34 may stop the pump 42. At step 1312, the controller 34 may determine whether a new setpoint has been selected by the user. If so, the controller 34 may return to step 1306. Otherwise, the controller 34 may proceed to step 1314 to determine if there has been a change in the measured blood pressure. If so, the controller 34 may return to step 1308 and run the pump 42 again to inflate or deflate the balloon 16 accordingly to return the blood pressure to the selected setpoint. If not, the controller 34 may await a new target setting at step 1316, and periodically return to step 1312.

Although the autonomous and manual modes of operation are described herein, the catheter 10 is not limited to being operated in these two distinct modes or in any particular modes with set operating steps, methods or techniques. The autonomous mode may include operation using a closed-loop control algorithm stored in the control hub 18. The autonomous mode may include various sub-modes, such as a setpoint mode, a target range mode, a set and hold mode or various additional modes that may be considered automatic modes. In the setpoint mode (as described above), the user may enter or the controller 34 may automatically set a single setpoint value of pressure or another parameter and the controller 34 directs inflation and/or deflation of the occlusion balloon 16 to match and maintain the pressure or other parameter in range of the setpoint value. The controller 34 may assign an acceptable range based on the selected setpoint value. For example, if the user selects a below balloon setpoint of MAP at 33 mmHg, the controller 34 may consider any measurement values within +/−2 mmHg of this setpoint (i.e., 31-35 mmHg) to be acceptable and a balloon volume change will only be made when the measured below balloon MAP is outside of this range. This prevents the controller 34 from making unnecessary changes due to natural oscillations in blood pressure from inherent impacts such as respiration or the like. In alternative embodiments, in addition to selecting a setpoint value, the user may also manually set the tolerance range around that setpoint value. A target range mode is similar to the setpoint mode, but instead of selecting a single value, the user or the controller 34 selects a range of values within which to maintain the pressure or other parameter.

FIG. 14 shows an example method 1400 for execution by the controller 34 when the user wishes to fully occlude the aorta. At step 1402, the controller 34 may receive a command to start full balloon 16 inflation, such as by long pressing the positive adjustment key 1003b on the display 30, although inputs for this command from other sources may be used as well. At step 1404, the controller 34 may begin running the pump 42 to inflate the balloon 16. At step 1406, the controller 34 may receive a blood pressure reading from the below-balloon blood pressure sensor 24. At step 1408, the controller 34 may compare the received below-balloon blood pressure reading with a predetermined limit (e.g., 4 mmHg, although the amount and units may differ as necessary). If the controller 34 finds the limit has been reached, at step 1410, the controller 34 may stop running the pump 42. At step 1412, the controller 34 may also output an alert to the user, such as through the display 30 or the like, that full occlusion has been achieved. If, on the other hand, the controller 34 at step 1408 determines the blood pressure limit has not been reached, the controller 34 may move to step 1414 and determine whether the current balloon pressure, as measured by the balloon pressure sensor 51, is less than a predetermined safety valve cracking pressure or other safety parameter. If so, the controller 34 may continue to run the pump 42. Otherwise, the controller 34 may move to step 1416 to stop running the pump 42. At step 1418, the controller 34 may output an alert to the user, such as through the display 30 or the like, that full occlusion could not be achieved and that the balloon is inflated to the saftest extent.

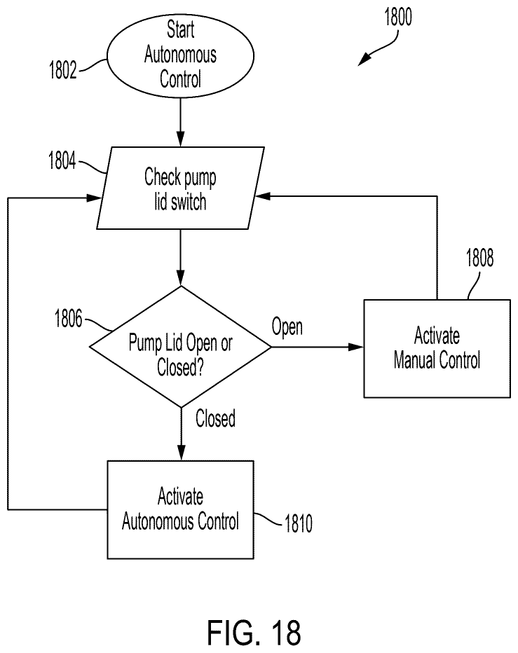

FIG. 18 shows an example method 1800 for execution by the controller 34 for switching between autonomous and manual control through operation of the pump lid 42a. At step 1802, the controller 34 may receive a command to autonomously maintain control of a blood pressure setpoint or setpoints, as previously described. At step 1804, the controller 34 may query the pump lid sensor 25. Based on the signal received from the pump lid sensor 25, at step 1806, the controller 34 may determine whether the pump lid 42a is open or closed. If the controller 34 finds the pump lid 42a open, at step 1808, the controller 34 may activate manual control and prevent operation of the pump 42. The controller 34 may return to step 1804 and continue polling the pump lid sensor 25. If the controller 34 finds the pump lid 42a to be closed, at step 1810, the controller 34 may activate autonomous control and allow operation of the pump 42 and proceed to autonomous operations as described above. However, the controller 34 may continually return to step 1804 to check that the pump lid 42a remains closed during autonomous operation. If the pump lid 42a is opened during autonomous control, the controller 34 may proceed to step 1808 and activate manual control.

Referring to FIG. 9, the control hub 18 in some embodiments may connect with the catheter 10 by way of a catheter hub 89. For example, the control hub 18 may be provided with a USB-C port 23b or other similar type of data port. A communication cable 91 (e.g., a USB cable, fiber-optic cable, or the like) may connect to the port 23b and to the catheter hub 89. Sensor data measured at the catheter 10 (e.g., data described above, such as blood pressure data, physiological parameters, and the like) may be collected at the catheter hub 89 for display and/or communication to the control hub 18 via the communication cable 91 (although wireless communication is also an option). The control hub 18 further may communicate with a traditional vital sign monitor 93. For example, the control hub 18 may be provided with a USB-C port 23c or other similar type of data port. A communication cable 95 (e.g., a USB cable or the like) may couple to a signal converter 97 that can receive the vital sign data from the control hub 18 and convert that data to the necessary protocols for receipt and display by the vital sign monitor 93. In this manner, the control hub 18 may be able to universally communicate with different types of vital sign monitors without having to be specifically configured for each particular type.

In some embodiments, the control hub 18 may use the pump 42 to fully deflate the balloon 16 to facilitate removal of the catheter 10 from the patient. FIG. 12 shows an example method 1200 for execution by the controller 34 for full balloon 16 deflation. At step 1202, the controller 34 may run the pump 42 to drain fluid from the balloon 16 back toward the reservoir 32. At step 1204, the controller 34 may periodically query the balloon pressure sensor 51 for a balloon pressure reading. At step 1206, the controller 34 may evaluate the received balloon pressure reading as compared with a target vacuum value (e.g., a predetermined pressure typically between 0 and atmospheric pressure, which may be in units of PSI or the like) or within a tolerance or range thereof suitable for removing the balloon 16 from the patient. If the condition is not met, the controller 34 may continue to run the pump 42. If the condition is met, the controller 34 may stop the pump 42 at step 1208. At step 1210, the controller 34 may output an alert, such as via the display 30 or the like, that full deflation has been achieved so the user is prompted to begin withdrawal of the catheter 10 from the patient.

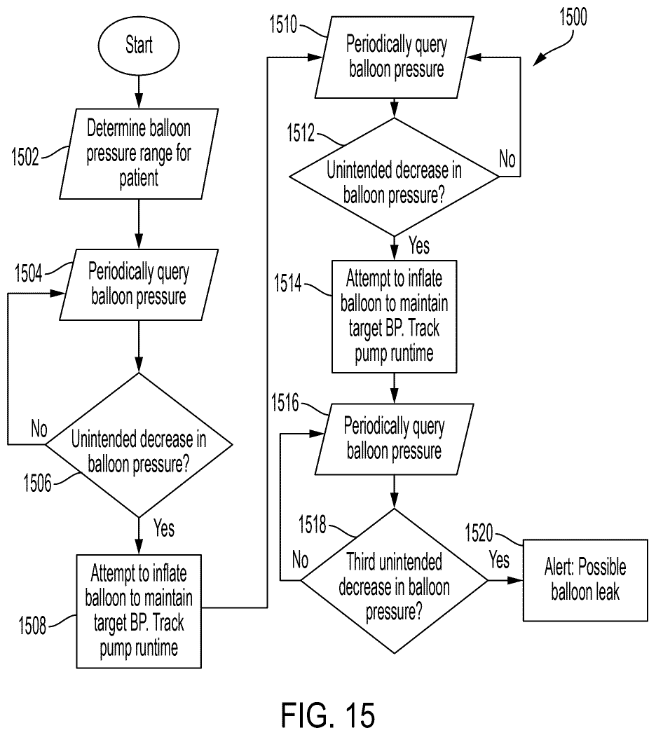

In some embodiments, the controller 34 may be configured to detect a possible leak in the balloon 16. FIG. 15 shows an example method 1500 for execution by the controller 34 for balloon leak detection. At step 1502, during operation, the controller 34 will determine an internal balloon pressure range for the patient based on readings from the balloon pressure sensor 51. At step 1504, the controller 34 will continue to periodically query the balloon pressure sensor 51 for an internal balloon pressure reading. At step 1506, if no unintended decrease in the balloon pressure outside of the previously determined pressure range is detected, the controller 34 simply continues to monitor. If an unintended decrease is detected, at step 1508, the controller 34 may attempt to inflate the balloon 16 using the pump 42 to maintain the target blood pressure setpoint(s). The controller 34 will also track pump 42 runtime. At step 1510, the controller 34 will continue to query the balloon pressure sensor 51. At step 1512, if no second unintended decrease in the balloon pressure outside of the previously determined pressure range is detected, the controller 34 simply continues to monitor. If a second unintended decrease is detected, at step 1514, the controller 34 may again attempt to inflate the balloon 16 using the pump 42 to maintain the target blood pressure setpoint(s) and track pump 42 runtime. At step 1516, the controller 34 will continue to query the balloon pressure sensor 51. At step 1518, if no third unintended decrease in the balloon pressure outside of the previously determined pressure range is detected, the controller 34 simply continues to monitor. If a third unintended decrease is detected, at step 1520, the controller 34 may output an alert, such as via the display 30 or the like, to notify the user of a possible leak in the balloon 16. Moreover, in the event the balloon 16 ruptures, the controller 34 may observe a fast and sudden decrease in balloon pressure and any attempts to reinflate the balloon 16 would likely result in no change in the balloon pressure. The controller 34 may then output an alert, via the display 30 or the like, to the user notifying of a potential rupture.

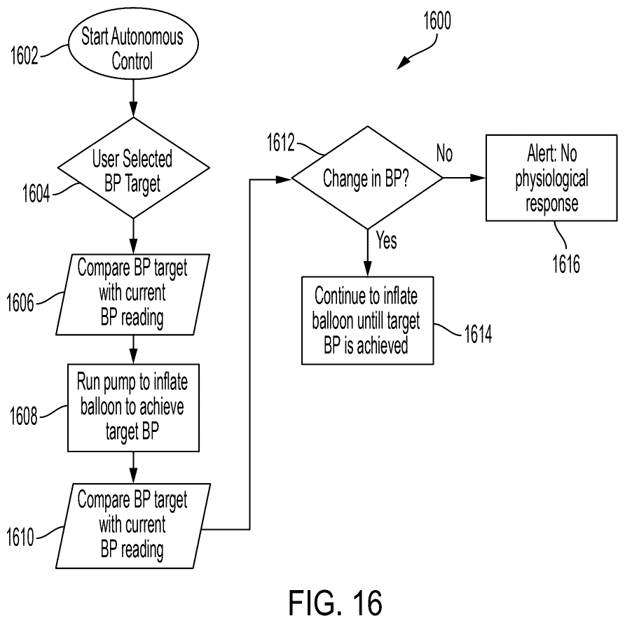

In some embodiments, the controller 34 may be configured to output an alert when the patient is not exhibiting a physiological response during an inflation cycle. FIG. 16 shows an example method 1600 for execution by the controller 34 for outputting such an alert. At step 1602, the controller 34 may enter the autonomous control operation and at step 1604 may receive a user-selected blood pressure setpoint. At step 1606, the controller 34 may compare the setpoint to a current blood pressure reading provided by at least one of the blood pressure sensors 22, 24. At step 1608, the controller 34 may run the pump 42 to inflate the balloon to achieve the blood pressure setpoint. At step 1610, the controller 34 may check the current blood pressure reading from at least one of the blood pressure sensors 22, 24 and compare it again with the blood pressure setpoint. At step 1612, the controller 34 may determine whether there has been a change in the blood pressure. At step 1614, if the blood pressure has changed, the controller 34 may continue to inflate the balloon 16 until the blood pressure setpoint is achieved. However, the controller 34 may continue to monitor for lack of changes in step 1612. At step 1616, if the blood pressure does not change in response to the balloon 16 inflation, the controller 34 may then output an alert, via the display 30 or the like, to the user that no change was detected to the patient's blood pressure. In addition, the controller 34 may also stop the pump 42 if no blood pressure change is detected in order to prevent an overinflation of the balloon 16.

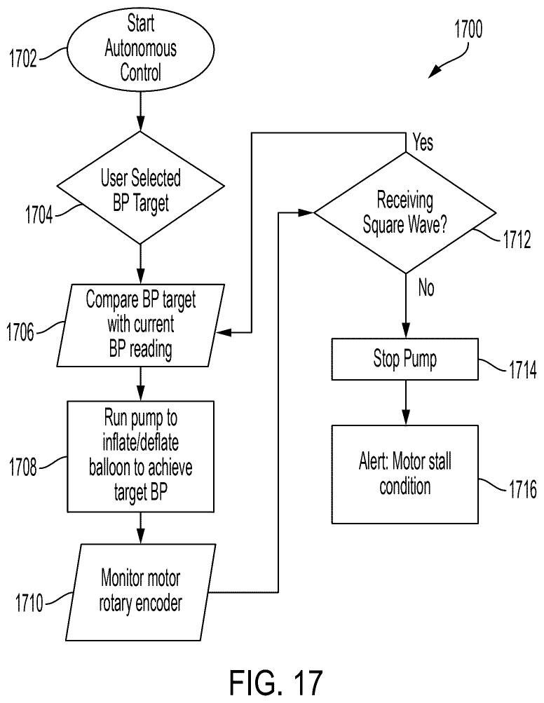

In some embodiments, the controller 34 may be configured to output an alert when the motor of the pump 42 stalls. FIG. 17 shows an example method 1700 for execution by the controller 34 for outputting such an alert. At step 1702, the controller 34 may enter the autonomous control operation and at step 1704 may receive a user-selected blood pressure setpoint. At step 1706, the controller 34 may compare the setpoint to a current blood pressure reading provided by at least one of the blood pressure sensors 22, 24. At step 1708, the controller 34 may run the pump 42 to inflate or deflate the balloon to achieve the blood pressure setpoint. At step 1710, the controller 34 may monitor the motor encoder 27. At step 1712, if the controller 34 determines that it is receiving a square wave or other signal from the motor encoder 27 indicative of normal motor operation, the controller 34 may continue to operate to achieve the blood pressure setpoint. If the controller 34 determines at step 1712 that a square wave or other appropriate signal is not being received, the controller 34 may, at step 1714, stop the pump 42. At step 1716, the controller 34 may then output an alert, via the display 30 or the like, to the user that the motor has stalled.

The controller 34 may include or be in communication with a processor module (not shown), which may be a Bluetooth low energy (“BLE”) processor module or a processor module utilizing other wireless protocols, such as ANT, Zigbee, Near Field Communication (NFC) and related protocols. The controller 34 may be in wireless, although not limited and may be wired, connection with a central hub (not shown) to transmit or cast acquired data from the sensors 22, 24, operation of the driver 38, motor, and pump 42, system status, warnings, alerts and other information or data generated by the controller 34 during use.

The controller 34 may conduct a device “handshake” via wired/wireless connection before operation can begin. The controller 34 may check connection with the distal and proximal sensors 22, 24, communication with the central hub, status of the batteries 56, volume of fluid in the reservoir 32, communication with the motor, communication with the pump 42, operation of the hub display 30, and/or the like, and may check various additional components and systems. In some embodiments, the catheter 10 also preferably conduct a “handshake” with an optical device before start of operation is granted. The catheter 10 may further include a balloon lumen, and/or a flow sensor therein that acquires flow data from the catheter balloon lumen and transmits the data to the controller 34. Flow sensors may also be otherwise mounted to the catheter 10 to detect internal flow of occlusion fluid or external flow of blood flow relative to the catheter during use, which flow data may be transmitted to the controller 34 and subsequently to the hub display 30 for representation on the hub display 30.

In some embodiments, the controller 34 and the hub housing 28 may also include speaker (not shown) and/or audio capability that permits audio messaging to the user. The controller 34 may, for example, be configured to “talk” the user through various procedural steps involved in the REBOA or P-REBOA process. The audio prompts may also be utilized to provide warnings to the user regarding system functionality, such as battery power, time limits for full occlusion, excessive blood pressure detection and related system or patient status.

In some embodiments, the hub housing 28 may be connected directly to the proximal shaft of the catheter 10 or may include a Luer fitting (not shown) or other connection fitting for connection of medical tubing to facilitate flow of occlusion fluid to and from the occlusion balloon 16. For example, the control hub 18 may be releasably connected to the proximal shaft of the catheter 10 by an electrical connection or pigtail cable. The electrical connection or pigtail cable may include a single lead, a double lead or additional leads, depending on designer preference and, potentially, the number of sensors 22, 24 that are in communication with the hub housing 28 and the controller 34. The control hub 18 may alternatively be electrically remote from the catheter 10 for communication between the controller 34, the sensors 22, 24 and the motor and pump 42 via wireless communication.

In some embodiments, the hub housing 28 may also be designed and configured for intravenous pole (“IV-pole”) mounting or tabletop/stand mounting. As a non-limiting example, the hub housing 28 may include a hook or loop to facilitate hanging from the IV-pole or an integral or separate clamp for mounting to a table or hospital bed. The hub housing 28 may also be strapped or otherwise mounted to a patient's extremity, using strap, suture, or adhered using strong medical grade adhesive, and may be positioned and adjusted to fit the contour of the patient's extremity by placing a curved surface thereof against the patient's extremity.

In embodiments where the batteries 56 are rechargeable, the controller 34 may be designed and configured for power management and management of the power of the batteries 56. Such battery packs 56 may be charged externally at a charging station or may alternatively be retained in the hub housing 28 and wired to a power source for recharging. The batteries or battery packs 56 may be placed on a dock or cradle (not shown) to be recharged or may be retained within the hub housing 28 for direct recharging when the control hub 18 is connected to a power source for recharging. The control hub 18 may alternatively be placed within a wireless charging environment to recharge its internal batteries 56. The control hub 18 may further be plugged directly into a wall outlet for recharging the batteries or battery packs 56. Alternatively, the control hub with display and electronics 18 may be AC-powered with no internal batteries 56.

In some embodiments, the control hub 18 may be configured to provide a status of the system and/or warnings to the user via the hub display 530. As a non-limiting example, the hub display 30 may utilize a color system to indicate to the user that the catheter 10 is operating within normal and acceptable ranges, such as by providing a green background in sections of the hub display 30 where acquired parameters of the system are within predetermined normal and acceptable ranges, such as a predetermined blood pressure range measured by the proximal pressure sensor 24 or an acceptable pressure within the occlusion balloon 16. Such a green indication may represent to the user that no changes to the system are required. Conversely, when parameters fall outside of predetermined acceptable ranges, the hub display 30 may illuminate a background of portions of the hub display 30 in red, orange or yellow to indicate that parameters are outside of acceptable levels and changes to the system are desired or required. The color coding of the hub display 30 and the maintenance of the acceptable ranges are preferably controlled or stored by the controller 34. As a non-limiting example, the hub display 30 may display a red background when too little flow is passing to the occlusion balloon 16 or lactate and pH are in an unacceptable range or fall outside of the predetermined acceptable range stored in the controller 34. The hub display 30 may include a representation of the shaft of the catheter 10 and the occlusion balloon 16 thereon with zones or boxes representing different portions of the shaft and occlusion balloon 16. The hub housing 28 may be connected to the proximal shaft of the catheter 10. The controller 34 may actuate the hub display 30 to turn boxes in potential problem areas or areas where acquired data is outside of the predetermined ranges red or orange, depending on how far outside of the predetermined ranges the acquired data falls and for how long.

In some embodiments, the hub housing 28 may be removably connected to the patient with a strap that is preferably connected to the housing 28 with hinges. The strap may include a buckle for releasable attachment to the patient, such as to the patient's leg during operation. The fluid reservoir 32 in this example may be a strap fluid reservoir that contains the fluid for inflating and deflating the occlusion balloon 16.

In some embodiments where the fluid reservoir 32 is integral to the hub housing 28, the fluid reservoir 32 may have a housing and can contain an internal bag or other container filled with inflation media. The bag may at least partially be made of ethylene vinyl acetate (EVA), polyethylene vinyl acetate (pEVA), polypropylene (PP), or similar, non-polyvinyl chloride (PVC) materials. The bag may include a tube (not shown) for allowing fluid to enter/exit the bag. The tube may be made from a non-PVC or non-Di(2-ethylhexyl) phthalate (DEHP) material that can attach to the bag via thermoforming or high-frequency welding techniques. In some embodiments, the fluid reservoir housing may store the inflation media itself without a further internal container. A sensor printed circuit board assembly (PCBA) may be used for the control hub 18 to detect that the fluid reservoir housing is connected to the control hub housing 28, as will be explained in further detail below.

In some embodiments, the fluid reservoir 32 may be non-detachable from the hub housing 28. Such fluid reservoir 32 may include an internal bag or other container that can be filled with inflation media via a syringe, an IV bag, or the like. To accommodate the syringe or other instrument for filling the fluid reservoir 32, a Luer port, an extension tube, and/or the like may be provided.

In some embodiments, the control hub 18 with the prefilled fluid reservoir attached may be sterilizable in its packaging. Suitable sterilization methods may include e-beam, gamma irradiation, ethylene oxide (ETO or EO), hydrogen peroxide plasma, or the like. In other embodiments, the control hub 18 may be sterilizable separately from the fluid reservoir 32. For example, the fluid reservoir 32 may be sterilized using e-beam or gamma irradiation while the control hub 18 is sterilized using ETO or hydrogen peroxide plasma.

In embodiments where the fluid reservoir housing is attachable to the hub housing 28 prior to use, the control hub housing 28 and the fluid reservoir housing can utilize mechanical latching features to secure to one another and prevent improper attachment. For example, snap fittings may be provided for mating the control hub housing 28 and the fluid reservoir housing. Magnetic fittings may be provided instead. Other types of mechanical attachment features may be used as well.

In one such configuration, the control hub 18 may be configured to detect that the fluid reservoir housing has been installed. As described above, the fluid reservoir housing may have a sensor PCBA coupled thereto. The sensor PCBA may be received in a card edge connector on the control hub housing. The card edge connector may be electrically connected with the controller 34 and function much like a switch. When the fluid reservoir housing is not attached to the control hub housing 28, the switch would remain in the normally open position and therefore let the controller 34 know. Conversely, when the fluid reservoir housing is fully seated into the hub housing 28, the switch would close, and the system would recognize the change. Symmetrical alignment of sensor PCBA pins to the card edge connector may be achieved by mechanical alignment pins. Other pins on the sensor PCBA could be used to energize the device, e.g., the controller 34 will not power up, until the fluid reservoir housing is fully inserted. This design is also helpful, as it would prevent the battery from being drained while in storage, and it keeps the device in a deenergized state during the sterilization process.

When the sensor PCBA of the fluid reservoir housing is used as the electrical interface, some pins could be dedicated as a power path from the battery supply to the remaining electronics of the control hub 18, such as the controller 34. When the fluid reservoir housing is not present, the power path would be open and conversely, when the fluid reservoir housing is snapped in place, the power path would be closed, providing power to the system.

In another configuration for fluid reservoir housing detection, one or more detector switches may be located on the control hub housing 28 and positioned to actuate in contact with the fluid reservoir housing. The detector switches may be connected to the controller 34. The detector switches may change logic states between HIGH and LOW based on the activation by attachment/detachment of the fluid reservoir housing. Two detector switches can be located at symmetrical locations to get activated when properly installed. Therefore, uneven or improper installation might not activate both detector switches with the same logical state and not cause the unit to power up and/or supply information to the controller 34.

In some embodiments, the control hub 18 may include a housing 28 that attaches to a catheter hub 89 in a clamshell fashion or other type of split design. The pump 42 may be housed in the control hub 18 such that when the control hub housing 28 is closed to the catheter hub 89, the pump 42, which may be a peristaltic pump, may engage with the medical tube 62 connecting a syringe 32 or other fluid reservoir or source (e.g., a saline bag or the like, not shown). As with the embodiment in FIGS. 1-6, the medical tube 62 may be received in a slot or channel within the control hub housing 28. The control hub 18 may be attached or closed to the catheter hub 89 prior to insertion of the catheter 10 into the patient. Alternatively, the control hub 18 may be attached or closed to the catheter hub 89 after the catheter 10 is inserted and partial occlusion has been established manually. For example, the user may manually inflate the balloon 16 using the syringe 32, then place the control hub 18 into an automated mode whereby the pump 42 can thereafter adjust inflation levels as appropriate. This may require only small adjustments to maintain the setpoint or range; however, the automatic algorithm may be designed to adjust to gross changes needed to maintain a steady target range and not to do excessive fine adjustments as parameters monitored experience minor variation as patient resuscitation dynamics change. In embodiments where the control hub 18 is only required to make minor adjustments to the balloon 16, it may be possible to power the control hub 18 from the catheter hub 89, depending upon other components utilized in the control hub 18 (e.g., the control hub 18 may include its own display, circuit board, and/or the like). In an alternate embodiment, the control hub 18 and catheter hub 89 may be combined in a single housing (not shown) with the pump 42. Rather than having a split design, the control hub 18 may include the slot or channel. The user may press the medical tube 62 into the slot or channel to engage with the pump 42.

In some embodiments, the control hub 18 may be able to move the pump 42 into and away from the medical tube 62. In this manner, the control hub 18 may be shipped with the pump 42 disengaged from the medical tube 62 to avoid plastic deformation of the tubing during transit and storage. This also allows the user to inflate or deflate the balloon (not shown) manually with the syringe 32 until an autonomous mode of operation is desired, in which case the control hub 18 can take over control of the balloon 16 using the pump 42. The user may also disengage the autonomous mode quickly by releasing the pump 42 from the medical tube 62 for manual control. The hub housing 28 may include a manual switch that can be physically actuated by the user for moving the pump 42 into the desired configuration relative to the syringe medical tube 62. The switch may also initiate manual and autonomous modes of operation, or such control may be provided separately, such as by a mode switch or via a touchscreen display 30.

In a similar embodiment, the pump 42 may be moved relative to the medical tube 62 based on signals from the controller 34. For example, the user may select an autonomous mode of operation or a pump movement action on a touchscreen display 30, and the controller 34 may then move the pump 42 into contact with the medical tube 62 for moving fluid from the syringe 32 or other fluid reservoir or source (e.g., a saline bag or the like, not shown).

In some embodiments, a control hub 18 may be selectively connected to multiple fluid sources 32, such as a syringe and a saline bag, although other types of fluid sources or reservoirs can be used in place of or in addition to those shown. For example, the syringe may be simple to use for manually inflating or deflating the balloon 16 and saline-filled syringes are readily available. However, it may be that friction of the plunger in the syringe is too great for the pump 42 to adequately push or pull fluid during an autonomous mode of operation. To avoid having to disconnect the syringe in favor of another type of fluid reservoir, like the saline bag, a three-way stopcock may be provided that can be manually or electronically actuated to select between the syringe and the saline bag as the fluid source for the catheter 10. The stopcock may have an embedded pressure relief valve, similar to that shown and described in International Patent Application Publication No. WO2022/016109, the entire contents of which are incorporated herein by reference. In embodiments where the stopcock is electronically actuated, the actuation may be made in response to a selection by the user via the touchscreen display 30 of an autonomous mode of operation, for example.

In some embodiments, the control hub 18 and catheter hub 89 may be combined in a single housing to provide the functionality described above.

Those skilled in the art will recognize that boundaries between the above-described operations are merely illustrative. The multiple operations may be combined into a single operation, a single operation may be distributed in additional operations and operations may be executed at least partially overlapping in time. Further, alternative embodiments may include multiple instances of a particular operation, and the order of operations may be altered in various other embodiments.

While specific and distinct embodiments have been shown in the drawings, various individual elements or combinations of elements from the different embodiments may be combined with one another while in keeping with the spirit and scope of the invention. Thus, an individual feature described herein only with respect to one embodiment should not be construed as being incompatible with other embodiments described herein or otherwise encompassed by the invention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1-16. (canceled)

17. A control system for an occlusion catheter having an occlusion balloon to control partial occlusion of a vessel of a patient, the control system comprising:
a fluid reservoir containing a fluid for use with the occlusion catheter;
a fluid connector connectable to the fluid reservoir and to the occlusion catheter to provide fluid communication between the fluid reservoir and the occlusion catheter, the fluid connector having a pressure sensor integrated therein;
a pump configured to move the fluid between the fluid reservoir and the occlusion balloon via the fluid connector;
a controller in electrical communication with the pump and the pressure sensor of the fluid connector, the controller configured to:
i. receive data from the pressure sensor of the fluid connector, the data being indicative of a pressure within the occlusion balloon,
ii. receive data representing a physiological value from at least one sensor located on or in the patient, and
iii. in an autonomous mode of operation, control the pump to alter a size of the occlusion balloon by driving fluid from the fluid reservoir toward the occlusion balloon or withdrawing fluid from the occlusion balloon toward the fluid reservoir, the control of the pump being based, at least in part, on the data from the pressure sensor of the fluid connector and the received data representing the physiological value and including comparing the received data representing the physiological value with a setpoint value or target range such that the size of the occlusion balloon is altered to cause the physiological value to be within a tolerance limit of the setpoint value or within the target range.

18. The control system of claim 17, wherein the physiological value is one of blood pressure upstream of the occlusion balloon or blood pressure downstream of the occlusion balloon.

19. The control system of claim 18, wherein the blood pressure upstream of the occlusion balloon is one of pulse pressure, systolic blood pressure, or mean arterial pressure and the blood pressure downstream of the occlusion balloon is one of pulse pressure, systolic blood pressure, or mean arterial pressure.

20. The control system of claim 17, wherein the control of the pump includes detecting a leak in the occlusion balloon, the controller being configured to output an alert when the controller determines the pressure in the balloon has unintentionally decreased at least once during operation based on the data from the pressure sensor of the fluid connector.

21. The control system of claim 20, wherein the controller is configured to output the alert when the controller determines the pressure in the balloon has unintentionally decreased more than twice during operation.

22. The control system of claim 17, wherein the control of the pump includes stopping the pump if the data from the pressure sensor of the fluid connector indicates the pressure in the balloon exceeds a predetermined safety value.

23. The control system of claim 17, wherein the control of the pump includes withdrawing fluid from the occlusion balloon in response to a command to empty the occlusion balloon, the controller being configured to stop the pump when the data from the pressure sensor of the fluid connector indicates a vacuum has been pulled in the occlusion balloon.

24. A control system for an occlusion catheter having an occlusion balloon to control partial occlusion of a vessel of a patient, the control system comprising:
a fluid reservoir containing a fluid for use with the occlusion catheter;
a fluid connector connectable to the fluid reservoir and to the occlusion catheter to provide fluid communication between the fluid reservoir and the occlusion catheter;
a pump configured to move the fluid between the fluid reservoir and the occlusion balloon via the fluid connector, the pump having a pump housing including a pump lid and a pump body, the pump lid being movable with respect to the pump body between an open position and a closed position, a portion of the fluid connector being received within the pump housing; and
a controller in electrical communication with the pump and a pressure sensor of the fluid connector, the controller configured to:
i. enter an autonomous mode of operation when the pump lid and pump body are in the closed position,
ii. in the autonomous mode of operation, receive data representing a physiological value from at least one sensor located on or in the patient, compare the received data with a setpoint value or target range, and control the pump to alter the size of the occlusion balloon to cause the physiological value to be within a tolerance limit of the setpoint value or within the target range,
iii. enter a manual mode of operation when the pump lid and pump body are in the open position, and
iv. in the manual mode of operation, prevent operation of the pump to enable movement of the fluid between the fluid reservoir and the occlusion balloon while the fluid connector is installed in the pump housing.

25. The control system of claim 24, wherein the physiological value is one of blood pressure upstream of the occlusion balloon or blood pressure downstream of the occlusion balloon.

26. The control system of claim 25, wherein the blood pressure upstream of the occlusion balloon is one of pulse pressure, systolic blood pressure, or mean arterial pressure and the blood pressure downstream of the occlusion balloon is one of pulse pressure, systolic blood pressure, or mean arterial pressure.

27. The control system of claim 24, further comprising a control hub housing, the controller being disposed within the control hub housing.

28. The control system of claim 27, wherein the pump is attached to the control hub housing.

29. The control system of claim 24, wherein the fluid reservoir is a syringe.

30. The control system of claim 24, wherein the pump body includes a Hall sensor and the pump lid includes a magnet, the controller being configured to determine the pump lid and pump body are in the closed position upon receiving a signal from the Hall sensor that indicates a presence of the magnet.

31. The control system of claim 24, wherein the controller is further configured to, upon determining from the data from the at least one sensor indicates no physiological response to a change in size of the occlusion balloon, output an alert.
